(19) United States
(12) Patent Application Publication
(10) Pub. No.: US 2021/0220162 A1
(43) Pub. Date: Jul. 22, 2021

(54) URINE EVACUATION DEVICE

(71) Applicant: Aron Jamison, Spokane, WA (US)

(72) Inventor: Aron Jamison, Spokane, WA (US)

(21) Appl. No.: 16/746,363

(22) Filed: Jan. 17, 2020

(51) Int. Cl. A61F 5/441; A61F 5/453

(52) U.S. Cl. CPC A61F 5/441; A61F 5/453

(57) ABSTRACT

A urine evacuation device includes an outer shell that is impermeable to liquid. The outer shell is formed of an elastic and deformable material such that the material redistributes upon compression and returns to an original shape when the compression is relieved. The outer shell is shaped to accommodate male genitalia therein. All peripheral edges of the outer shell are rounded. A port extends through a front side of the outer shell for urine evacuation. A liquid permeable layer disposed within the outer shell.

### BACKGROUND

Worldwide, every year, many millions of urinary catheters and other urinary evacuation devices (“evacuation device”) are used to assist in the evacuation of urine from persons who either cannot do so on their own for a myriad of reasons. The type of evacuation device used may vary depending sometimes on the situation and needs of the patient to whom the evacuation device is applied and sometimes on the availability of one or more types of evacuation devices. Nevertheless, the ability to evacuate urine from a person in an effective manner and as pain-free and safely as possible is an ongoing concern.

One particular concern is the rising number of patient infections that are traced back to the use of a urinary catheter and some other evacuation devices. Other issues, particularly with regard to the use of catheters that are inserted into the urinary tract, include: potential physical damage to the urinary tract, time wasted in unsuccessful efforts to insert the catheter, higher risk of infection, patient discomfort, etc.

While externally placed urinary evacuation devices are available, issues of comfort, effectiveness, and convenience remain. Accordingly, additional improvements in urinary evacuation devices are needed.

### BRIEF DESCRIPTION OF THE DRAWINGS

The Detailed Description is set forth with reference to the accompanying figures. In the figures, the left-most digit(s) of a reference number identifies the figure in which the reference number first appears. The use of the same reference numbers in different figures indicates similar or identical items. Furthermore, the drawings may be considered as providing an approximate depiction of the relative sizes of the individual components within individual figures. However, the drawings are not to scale, and the relative sizes of the individual components, both within individual figures and between the different figures, may vary from what is depicted. In particular, some of the figures may depict components as a certain size or shape, while other figures may depict the same components on a larger scale or differently shaped for the sake of clarity.

FIG. 1 illustrates a perspective front view of an external urine evacuation device according to an embodiment of this disclosure.

FIG. 2 illustrates a front view of the external urine evacuation device of FIG. 1 according to an embodiment of this disclosure.

FIG. 3 illustrates a side view of the external urine evacuation device of FIG. 1 according to an embodiment of this disclosure.

FIG. 4 illustrates a rear view of the external urine evacuation device of FIG. 1 according to an embodiment of this disclosure.

FIG. 5 illustrates a side cross-sectional view of the external urine evacuation device of FIG. 1 according to an embodiment of this disclosure.

### DETAILED DESCRIPTION

### Overview

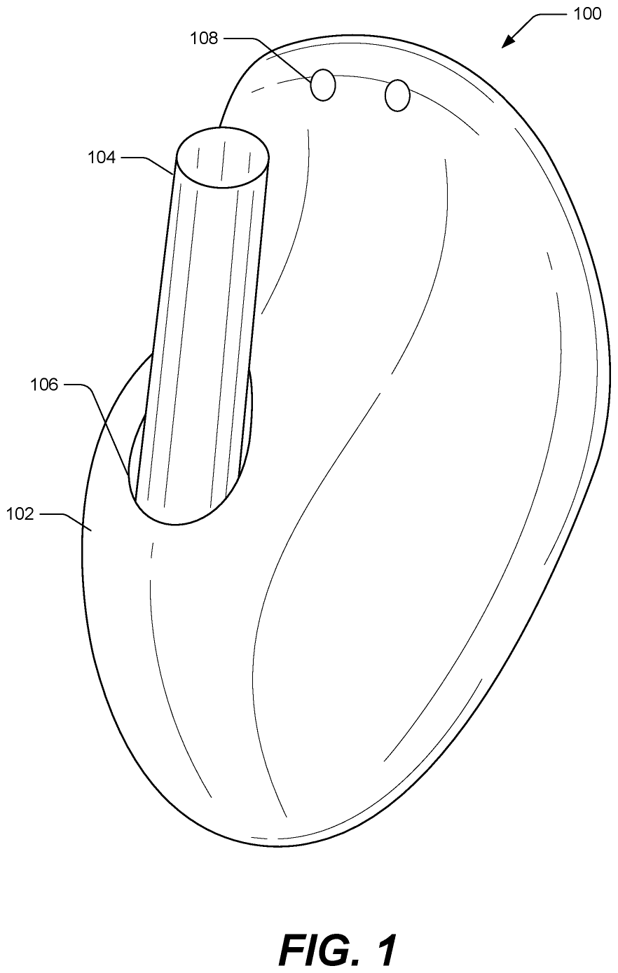

This disclosure is directed to an external urine evacuation device. In an embodiment, a urine evacuation device may be shaped as depicted in FIGS. 1-5, and such a shape may be particularly beneficial for use evacuating urine from male genitalia. It is contemplated, however, that other shapes not shown herein may be satisfactory as well for male genitalia, and that yet the same or other shapes may be similarly or more accommodating for female genitalia. To be clear, reduction and redistribution of the pressure from the device on the body on which the device is used are paramount factors for consideration. As such, the materials and specific shape of the urine evacuation device may vary for manufacturing, use, and effectiveness purposes, as well as for comfort of the individuals on whom the device is used. Nevertheless, the embodiment as depicted is considered to be more comfortable and effective for urine evacuation, at least from male genitalia.

In FIG. 1, a urine evacuation device 100 (“device 100”) is depicted in a perspective view from a direction looking toward the front side of the device 100. In an embodiment, device 100 may include an outer shell 102 that is impermeable to liquids, in particular, urine. Outer shell 102 secures a permeable layer (not shown in FIG. 1) therewithin. Device 100 further includes a tube 104 that passes through a port 106 in the depicted in the front side wall of outer shell 102. Port 106 extends through a thickness of outer shell 102 such that tube 104 is able to extend therethrough to be disposed with an end of tube 104 adjacent to the permeable layer. Additionally, one or more ventilation ducts 108 may be included as holes, for example, at an upper end of device 100. Notably, the one or more ventilation ducts 108 may assist in creating a pressure differential across the permeable layer for directional suction to retrieve urine.

Inasmuch as outer shell 102 is intended to be placed against the genitals, a material of outer shell 102 should be hypoallergenic and inert. Materials having properties akin to silicone are contemplated for outer shell 102. Silicone rubber is contemplated for use due to the flexibility, compressibility, durability (e.g., variable shore hardness, high tensile strength, and tear resistance), impermeability to urine, high-temperature resistance, elasticity and deformability, moldability, inert nature with respect to human skin, and soft, rubbery feel against flesh. These material properties are of particular significance when considering the frictional movement and pressure of device 100 against a patient's skin and muscles when the body shifts positions. For example, an elastic and deformable material allows superior pressure reduction and redistribution when compressed against a body.

Furthermore, a material selected for outer shell 102 may be chosen for the ability to quickly mass-produce in a mold having a shape with minimal or no angled surface intersections so as to eliminate any “corners.” That is, it is desirable for outer shell 102 to have curved or rounded edges wherever possible to minimize impact of device 100 on a patient's body. In an embodiment, materials such as silicone may be used to form outer shell 102, and device 100 may be formed via liquid injection molding, for example. A material hardness of the evacuation device may vary widely. However, in an embodiment, the material selected may have a durometer range between a shore OO scale to a shore A scale, (e.g., shore 00 hardness of 10 A or 15 A, for instance. In other embodiments, the shore hardness of the material may be more than 15 A, (e.g., up to about 90 A) or even less than 10 A (e.g., as low as about 30OO) for example. Moreover, it is noted that port 106 may be molded directly in outer shell 102 or may be punched out after molding outer shell 102. Similarly, tube 104 may be placed in outer shell 102 during molding as a part of forming port 106, or tube 104 may be added after molding outer shell 102.

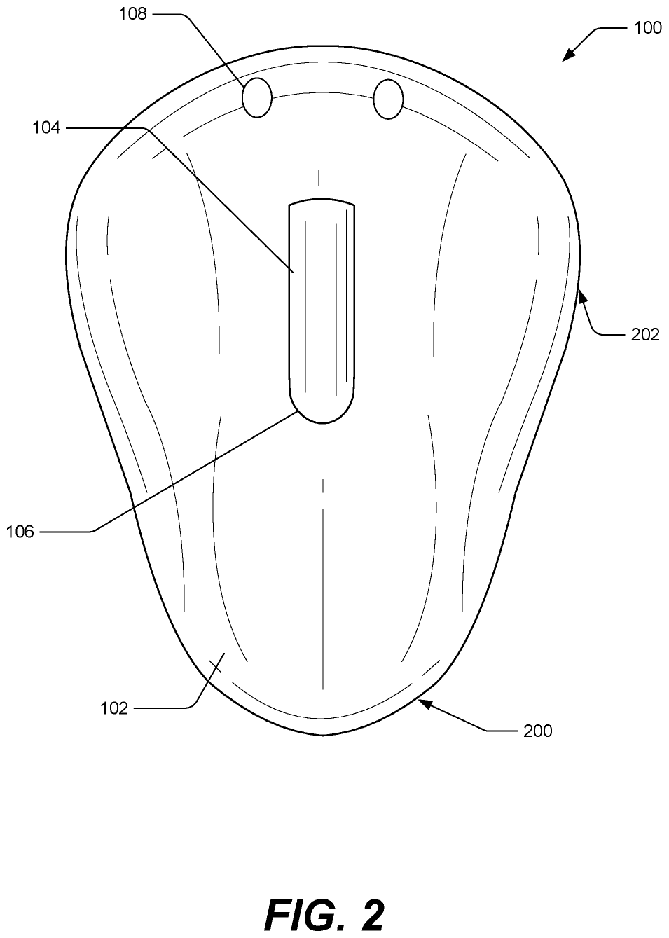

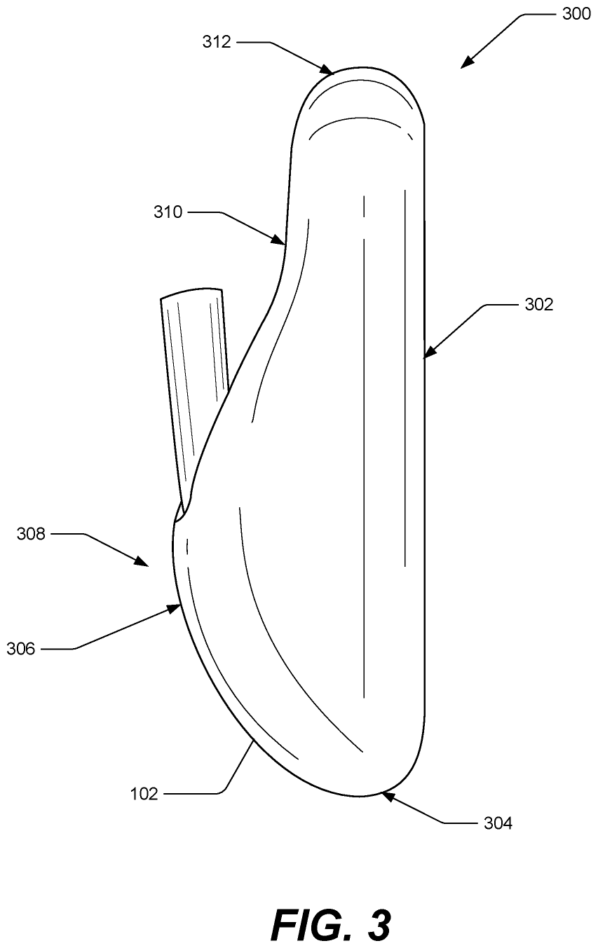

As depicted in FIG. 2, a peripheral shape of outer shell 102 of device 100 may be similar to an upside-down pear shape, having a narrower lower end 200 compared to the wider upper end 202. In FIG. 3, a profile 300 of outer shell 102 may be defined as a substantially planar back side 302 connected via a rounded lower edge 304 to a bulge 306 that protrudes outward on the lower portion of the front side 308 and then tapers to a relatively narrow neck 310 on the upper portion of the front side before rounding again at a top edge 312 going back to the planar back side 302. An advantage of the narrow neck is the reduction of material that could interfere in movement of the patient.

In further explanation of the above components with respect to the profile 300, port 106 may be formed in the bulge 306 such that a direction of an axis of the port extends at an acute angle with respect to a plane aligned with the substantially planar back side 302. As such, the portion of tube 104 that is inserted is fixed in a vertically extending position so as to lead tube 104 out of outer shell 102 in a direction that extends upward away from the lower body of the patient. This orientation may be beneficial to conform more easily with clothing worn by or coverings over the user.

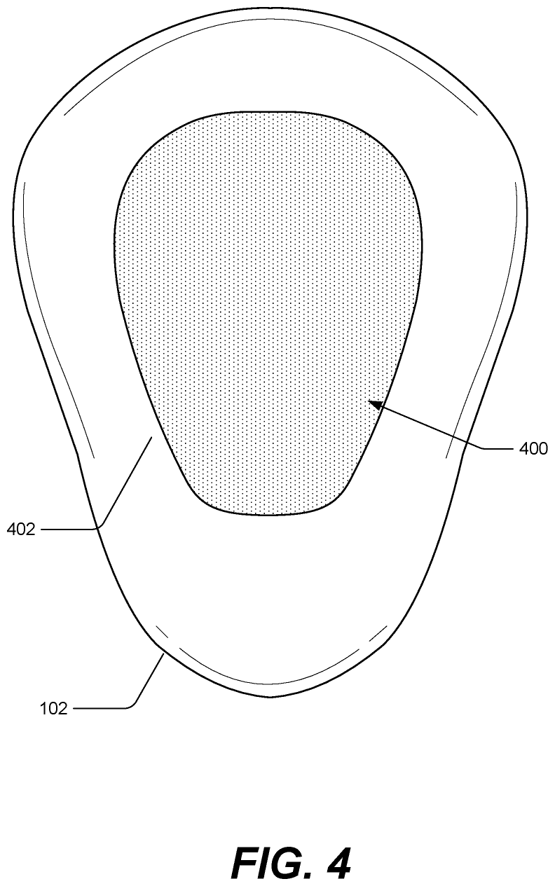

As mentioned above, outer shell 102 secures a permeable layer 400 (e.g., a sheet, a membrane, etc.) shown in FIG. 4 on the interior of outer shell 102. Outer shell 102 may further include a peripheral flange 402 that extends from an outer perimeter of outer shell 102 inwardly toward a central location on a plane of the back side of outer shell 102. Flange 402 may be continuous around the entire perimeter of outer shell 102 as shown or may have breaks therethrough (not shown). Permeable layer 400 may be a porous, soft material to allow urine to pass therethrough to be suctioned away from the body. In an embodiment, permeable layer 400 may be formed of a liquid wicking material to draw away moisture from the body. In an embodiment, permeable layer 400 may be an open-cell foam, such as a polyurethane foam, for example. Moreover, in an alternative embodiment, permeable layer 400 may be a membrane having permeability that works in only one direction. In such an embodiment, the permeable layer 400 may allow urine to pass through to be evacuated via tube 104 and prevent urine from remaining in large quantities against the skin of the user/patient. Furthermore, in an embodiment including the one or more ventilation ducts 108, as indicated above, by locating such openings in outer shell 102 opposite the end through which tube 104 enters outer shell 102, the orientation may assist in creating a pressure differential across the permeable layer 400 thereby creating a directional suction to retrieve urine.

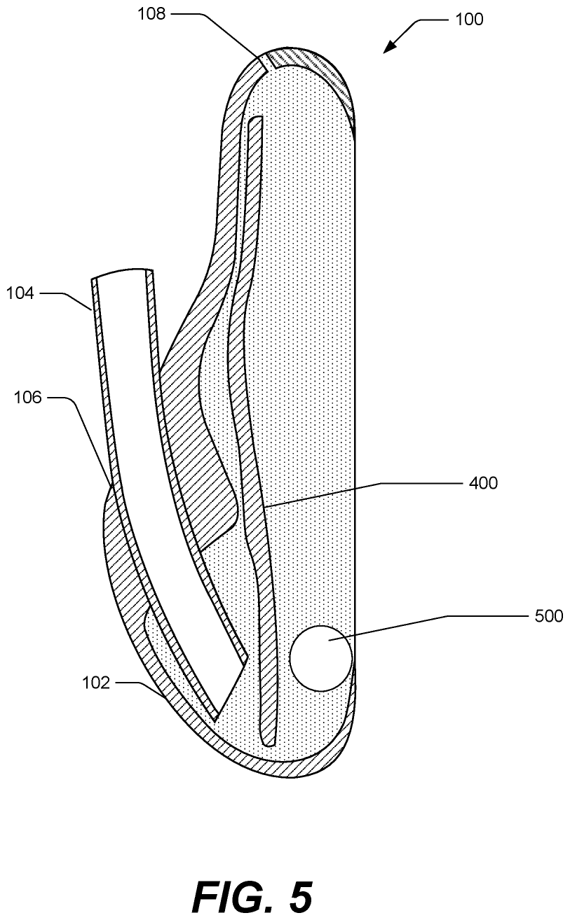

In FIG. 5, a cross-sectional view of device 100 is shown. Note, walls of outer shell 102 may be relatively thin, while maintaining the desired profile/shape of device 100. In an embodiment, permeable layer 400 may fit easily within outer shell 102 and may spread out evenly without compression. Alternatively, permeable layer 400 may be compressed to some extent to fit snugly inside outer shell 102 and within flange 402. In an embodiment, the compressed state of permeable layer 400 assists in maintaining a shape of outer shell 102.

In an embodiment, one or more moisture sensors 500 may be embedded in outer shell 102 to detect moisture and communicate with an associated vacuum pump (not shown) to activate the suction. A moisture sensor 500 may minimize the amount of time the suction is on, thereby reducing the risk of overly dry skin and causing sores on the body. Further, moisture sensor 500 may assist in adequately and timely suctioning urine upon release, thereby preventing the skin from being exposed to the moisture of the urine for excessive amounts of time and causing sores on the body. Moisture sensor 500 may be one of known types or a modification of a known type, including, but not limited to: infrared sensors, contact sensors, etc.

### CONCLUSION

Although several embodiments have been described in language specific to structural features and/or methodological acts, it is to be understood that the claims are not necessarily limited to the specific features or acts described. Rather, the specific features and acts are disclosed as illustrative forms of implementing the claimed subject matter.

What is claimed is:

1. A urine evacuation device comprising:
an outer shell that is impermeable to liquid, the outer shell being formed of an elastic and deformable material such that the material redistributes upon compression and returns to an original shape when the compression is relieved, the outer shell being shaped to accommodate male genitalia therein, and all peripheral edges of the outer shell being rounded;
a port extending through a front side of the outer shell for urine evacuation; and
a liquid permeable layer disposed within the outer shell.

2. The urine evacuation device according to claim 1, further comprising a tube fixed in the port.

3. The urine evacuation device according to claim 2, wherein an axis of the port extends at an acute angle with respect to a plane aligned with a back side of the outer shell such that the tube exits the outer shell in a direction upward with respect to a body of a user of the device.

4. The urine evacuation device according to claim 1, further comprising one or more ventilation ducts through an upper end of the outer shell.

5. The urine evacuation device according to claim 1, wherein the outer shell is a unitary molded piece.

6. The urine evacuation device according to claim 1, further comprising a flange that extends inwardly from the peripheral edge on a back side of the outer shell.

7. The urine evacuation device according to claim 1, wherein the material of the outer shell includes a material having a shore hardness in a range between shore 3000 and shore 90 A, and
wherein the permeable layer includes a polyurethane foam.

8. A urine evacuation device comprising:
an outer shell that is impermeable to liquid, the outer shell being formed of an elastic and deformable material such that the material redistributes upon compression and returns to an original shape when the compression is relieved, and all peripheral edges of the outer shell being rounded;
a port extending through a front side of the outer shell for urine evacuation;
a tube to evacuate urine from the inner shell, the tube extending from an interior of the outer shell through the port to extend upward from a bulge in a profile of a lower end of the outer shell, and the tube being molded into the outer shell so as to be fixed with the outer shell; and
a liquid permeable layer disposed within the outer shell.

9. The urine evacuation device according to claim 8, wherein the liquid permeable layer is in a partially compressed state within the outer shell.

10. The urine evacuation device according to claim 8, wherein the liquid permeable layer includes a moisture wicking material.

11. The urine evacuation device according to claim 8, further comprising one or more ventilation holes through the outer shell.

12. The urine evacuation device according to claim 8, further comprising a flange that extends inwardly from the peripheral edge on a back side of the outer shell.

13. The urine evacuation device according to claim 8, wherein a side profile of the outer shell is defined by:
a substantially planar back side that terminates at the lower end of the outer shell,
a rounded lower edge continuous with the back side,
the bulge that protrudes outward on the lower portion of a front side of the side profile of the outer shell, the bulge being continuous with the rounded lower edge,
a neck that is relatively narrow compared to the bulge, the neck extending upward from the bulge on an upper portion of the front side, and
a rounded top edge that is continuous between the neck and the back side.

14. The urine evacuation device according to claim 8, wherein the outer shell includes silicone and the permeable layer includes a polyurethane foam.

15. A urine evacuation device comprising:
an outer shell that is impermeable to liquid, the outer shell being formed of an elastic and deformable material such that the material redistributes upon compression and returns to an original shape when the compression is relieved, and all peripheral edges of the outer shell being rounded;
a tube to evacuate urine from the inner shell, the tube extending from an interior of the outer shell through the port to extend upward from a bulge in a profile of a lower end of the outer shell, and the tube being molded into the outer shell so as to be fixed with the outer shell;
a liquid permeable layer disposed within the outer shell; and
at least one ventilation duct through the outer shell.

16. The urine evacuation device according to claim 15, wherein the liquid permeable layer includes a moisture wicking material.

17. The urine evacuation device according to claim 16, wherein the liquid permeable layer includes a moisture wicking polyurethane foam.

18. The urine evacuation device according to claim 15, further comprising:
an opening at a back side of the outer shell; and
a flange that extends inwardly from an entire perimeter of the opening at the back side of the outer shell to assist in trapping urine therein for evacuation.

19. The urine evacuation device according to claim 15, wherein an axis of a portion of the tube disposed within a wall of the outer shell extends at an acute angle with respect to a plane aligned with a back side of the outer shell such that a portion of the tube external to the outer shell extends in a direction upward with respect to a body of a user of the device.

20. The urine evacuation device according to claim 15, wherein the at least one ventilation duct includes a pair of ventilation ducts spaced apart at an upper end of the outer shell.
